(19) United States
(12) Patent Application Publication
(10) Pub. No.: US 2012/0209140 A1
(43) Pub. Date: Aug. 16, 2012

(54) Tissue Sampling Device

(76) Inventor: Shawn Ryan, Upton, MA (US)

(21) Appl. No.: 13/210,061

(22) Filed: Aug. 15, 2011

Related U.S. Application Data
(60) Provisional application No. 61/373,944, filed on Aug. 16, 2010.

(51) Int. Cl. A61B 10/06; A61B 10/02

(52) U.S. Cl. 600/564; 600/562

(57) ABSTRACT

A biopsy device includes a longitudinal element sufficiently flexible to permit insertion into a body. The longitudinal element extends from a proximal end to a distal end and includes a lumen extending therethrough. The device also includes a stylet slidably received within the lumen having a distal end split into a pair of arms biased away from one another so that, when distal end of the stylet is advanced out of a distal end of the longitudinal element, the arms separate from one another into a tissue receiving configuration, the stylet including a tissue receiving space between the arms. The stylet is coupled to a handle via which the stylet is drawn proximally into the lumen of the longitudinal element so that contact between the longitudinal element and the arms draws the arms toward one another into a tissue capture configuration in which tissue is gripped therebetween.

### PRIORITY CLAIM

The application claims the priority to the U.S. Provisional Application Ser. No. 61/373,944, entitled “Tissue Sampling Device” filed Aug. 16, 2010. The specification of the above-identified application is incorporated herewith by reference.

### BACKGROUND

Needle biopsies are common for the diagnosis and the staging of disease. One type of biopsy procedure is endoscopic ultrasound-guided fine needle aspiration (EUS-FNA) which involves the insertion of a needle under ultrasound guidance so that the physician may accurately gauge the position of the needle relative to target tissue to be sampled. EUS-FNA procedures are performed to ensure that the correct tissue is sampled while minimizing risk to the patient. However, in certain situations, a sample may be difficult to handle or acquire. One type of device currently in use includes a notched stylet which is extended beyond a distal end of the needle to place the notch of the stylet within a target tissue mass. When the notch of the stylet is positioned as desired, a mechanism which may include a spring and/or gas operated piston, is released to drive the needle distally over the stylet capturing a sample within the notch. However, in certain situations, it may not be desirable to have the needle driven distally without direct physician control. Furthermore, such devices often fail to capture a sample. For example, when the needle extends through the body to the target tissue mass along a tortuous path, the energy released through actuation of the spring may dissipate before it reaches the distal end of the needle, reducing the suddenness of the distal movement of the needle over the stylet and pushing the target tissue out of the notch. Additionally, the column strength of certain needles is reduced to facilitates their insertion over tortuous paths. This may cause these needles to buckle when the mechanism is actuated attenuating the movement of the distal end of the needle over the stylet. This buckling may slow the distal movement of the needle over the stylet to a level at which tissue is pushed out of the notch instead of being sliced and maintained therein.

### SUMMARY OF THE INVENTION

The present invention is directed to a biopsy device, comprising a longitudinal element sufficiently flexible to permit insertion into a body via a natural body lumen, the longitudinal element extending from a proximal end to a distal end and including a lumen extending therethrough in combination with a stylet slidably received within the lumen having a distal end split into a pair of arms biased away from one another so that, when distal end of the stylet is advanced out of a distal end of the longitudinal element, the arms separate from one another into a tissue receiving configuration, the stylet including a tissue receiving space between the arms, wherein the stylet is coupled to a handle via which the stylet is drawn proximally into the lumen of the longitudinal element so that contact between the longitudinal element and the arms draws the arms toward one another into a tissue capture configuration in which tissue is gripped therebetween an actuator coupled to the stylet for withdrawing the stylet proximally relative to the longitudinal element from the tissue receiving configuration to the tissue capture configuration.

### BRIEF DESCRIPTION OF THE DRAWINGS

FIG. 1 shows a side view of a distal end of a device according to an exemplary embodiment of the present invention;

FIG. 2 shows a cross-sectional side view of the distal end of the device of FIG. 1, in an open configuration;

FIG. 3 shows a cross-sectional side view of the distal end of the device of FIG. 1, in a closed configuration;

FIG. 4 shows a side view of an actuating mechanism according to an exemplary embodiment of the present invention;

FIG. 5 shows a cross-sectional perspective view of the actuating mechanism of FIG. 4; and

FIG. 6 shows a perspective view of an alternate embodiment of a housing of the actuating mechanism of FIG. 4.

### DETAILED DESCRIPTION

The present invention may be further understood with reference to the following description and the appended drawings, wherein like elements are referred to with the same reference numerals. The present invention relates to devices for needle biopsies and, in particular, relates to an improved biopsy device including a mechanically actuated stylet for extracting target cells. Exemplary embodiments of the present invention describe a stylet movable between a tissue capture configuration in which the device collects a sample and a closed configuration in which the stylet is drawn proximally within a needle, covering the collected tissue sample such that the device may be removed from a body and the collected tissue sample examined. Although exemplary embodiments of the present invention describe an EUS-FNA device, it will be understood by those of skill in the art that the present invention may relate to any biopsy device configured for delivery to a target site within a body via insertion through an endoscope, spyscope, another needle or a catheter such as, for example, a biliary catheter, a balloon catheter, a stent delivery catheter, etc.

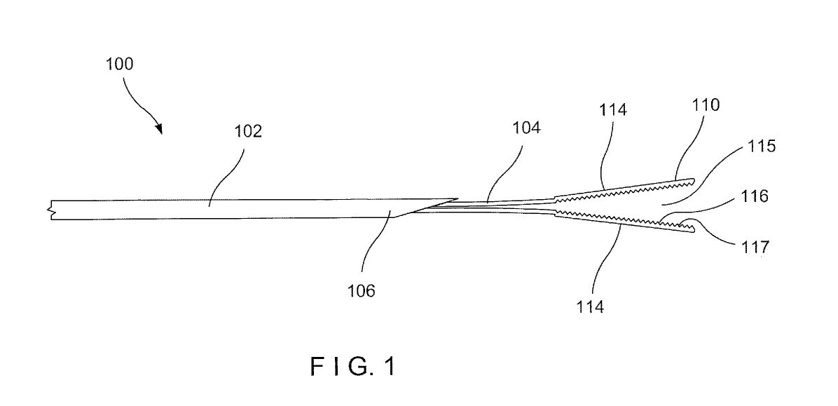

As shown in FIGS. 1-3, a device 100 according an exemplary embodiment of the invention comprises a needle 102 and a stylet 104 housed substantially within the needle 102 and movable between an open tissue receiving configuration and a closed tissue capturing configuration. The needle 102 extends longitudinally from a tissue penetrating distal tip 106 to a proximal end (not shown) with a lumen 108 extending therethrough. The needle 102 may be formed, for example, as a hypotube that is cut at an angle to form the tissue penetrating distal tip 106. The needle 102 may be formed of any suitable material such as, for example, a polymer, stainless steel, a chromium cobalt alloy, nitinol or any combination of such materials. For example, a distal portion of the needle 102 may be formed of a metal while a proximal portion of the needle 102 is formed of a polymer. In a preferred embodiment, the needle 102 is sized to navigate through torturous paths within the body. For example, an outer diameter of the needle may range from between 0.8 mm to 1.4 mm, and more preferably between 1.0 mm and 1.2 mm. It will be understood by those of skill in the art, however, that the needle 102 may be formed of any of a variety of materials and in any of a variety of sizes so long as the needle 102 is sufficiently flexible to navigate through the body to the site of the target tissue to be sampled while retaining the column strength necessary to be advanced distally as required to sample tissue.

The stylet 104 extends longitudinally within a lumen 108 of the needle 102 from a distal end 110 to a proximal end 112 and is slidable therewithin. The distal end 110 of the stylet 104 includes a pair of arms 114 for gripping tissue therebetween. The stylet 104 is formed of a material which is sufficiently flexible so that, when the stylet 104 is in the closed configuration, the needle 102 may be navigated along a tortuous path to a target tissue site. The stylet 104 may be formed of a material such as metal, plastic, other biocompatible materials or any suitable combination thereof. The stylet 104 may also be formed as a hypotube. In a preferred embodiment, the stylet 104 is formed of a nitinol wire cut longitudinally to include a channel 115 extending from the distal end 110 along a portion of a length thereof such that the distal end 110 is split into the pair of arms 114, which may grip tissue therebetween. It will be understood by those of skill in the art, however, that the stylet 104 may include any number of arms 114 so long as the arms 114 are movable relative to one another to grip tissue therebetween. In a preferred embodiment, the stylet 104 may have an outer diameter ranging from between 0.5 mm and 1.2 mm, and more preferably between 0.7 mm and 0.9 mm. It will be understood by those of skill n the art, however, that the stylet 104 may be formed of any size so long as the stylet 104 is insertable through the needle 102 and into a living body.

The stylet 104 is movable relative to the needle 102 such that the arms 114 may extend distally past the distal end 106 of the needle 102 into an open, tissue receiving configuration. The arms 114 are biased toward the open configuration by, for example, by being heat set. Alternatively, the arms 114 may be biased toward the open configuration by pre-bending the arms 114 at a hinged area at which the arms 114 meet. In a preferred embodiment the arms 114 may be angled relative to one another in an open, tissue receiving configuration. In an alternate embodiment, the arms 114 may be substantially parallel to one another and spaced apart to receive tissue therebetween. The pair of arms 114 may also include a tapered distal tip to aid in piercing tissue. The arms 114 may also treated and/or include features for facilitating gripping of tissue therebetween. For example, the arms 114 may include at least one tooth or, alternatively, a series of teeth 116 along inner surfaces 117 thereof, for gripping and holding tissue received therebetween. The arms 114 may also include a recess along the inner surfaces 117 thereof so that more tissue may be grasped between the arms 114. The inner surfaces 117 may also be treated with a coating for enhancing a grip between the arms 114.

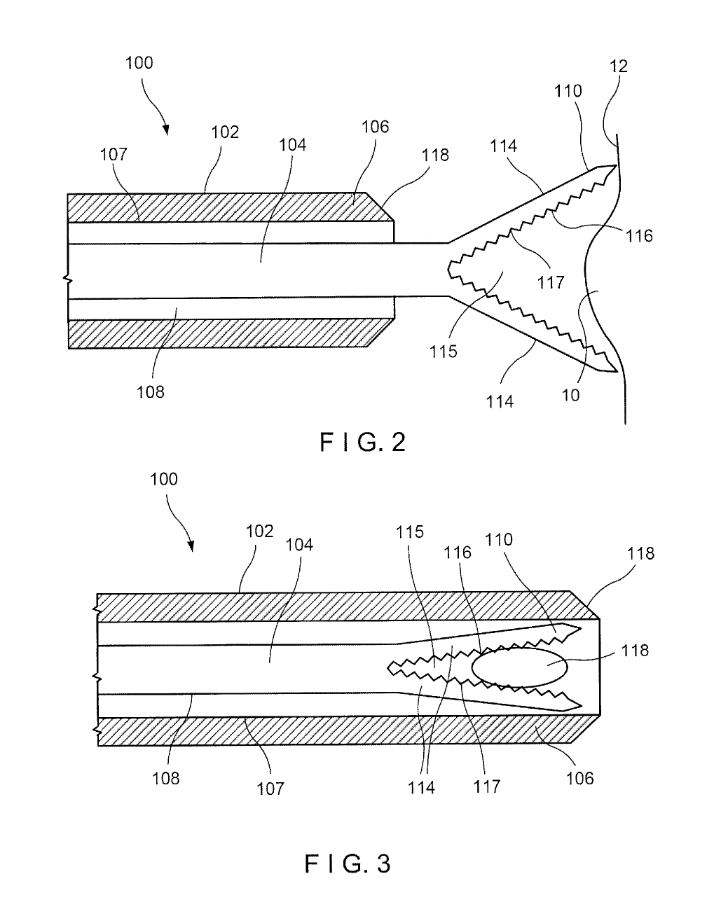

The device 100 is inserted into the body in the closed configuration to facilitate navigation to a target tissue site (e.g., along a tortuous path through natural body lumens). When the device 100 is in a desired position relative to a target tissue site, the device 100 is moved to the open configuration in which, as shown in FIG. 2, the distal end 110 of the stylet 104 is extended distally beyond the distal tip 106 of the needle 102 with the arms 114 outside the needle 102 adjacent to target tissue. When the arms 114 are outside the needle 102, they revert to the open configuration under their bias so that a tissue sample 10 may be received therebetween. The user then positions the arms 114 so that a desired portion of tissue 10 is received in the channel 115 between the arms 114. The stylet 104 is then drawn proximally through the lumen 106 of the needle 102 moving the arms 114 into the closed configuration as they reenter the needle 102. Specifically, as they are drawn into the needle 102, as shown in FIG. 3, an inner wall 107 of the needle 102 presses against the arms 114 moving them toward one another into the closed configuration trapping the portion of tissue 10 therebetween. As the stylet 104 is drawn proximally through the needle 102, the tissue sample 10 gripped between the arms 114 is cut away from the surrounding tissue 12 by a distal edge 118 of the needle 102 and retained within the lumen 106 for withdrawal from the body. It will be understood by those of skill in the art that the teeth 116 enhance the gripping of the tissue sample 10 resisting forces tending to draw the sample 10 out of the channel 115 as the stylet 104 is withdrawn into the needle 102. As the stylet 104 is moved proximally into the needle 102 to capture the tissue portion 116, axially compressive forces are applied to the needle 102 at the distal end while the forces applied to the stylet 104 are tensile. That is, tension is applied to move the stylet 104 while holding the position of the needle 102 substantially constant, minimizing buckling and other problems associated with devices that push a needle distally over a stylet to capture tissue. This allows the distal edge 124 of the needle 102 to more efficiently cut the tissue sample 10 from surrounding tissue 12. Although in the preferred embodiment the device 100 is inserted into a body lumen and the needle 102 is an FNA needle, it will be understood by those of skill in the art that the device 100 may be used to collect tissue samples in any target site within a living body such that the needle 102 may be any type of needle, catheter or tube insertable into a living body. Thus, it is respectfully submitted that any arrangement, shape, size, opening of arms 114, number and/or combination of teeth 116 or other gripping features are contemplated within the present invention.

Furthermore, the stylet 104 of this embodiment is drawn proximally through the needle under direct control of a user through a standard connection of the stylet 104 and needle 102 to a manual mechanism. For example, a handle (not shown) of the device may include a first member (not shown) connected to the stylet 104 with a second member coupled to the needle 102 so that movement of the first member proximally relative to the second member draws the stylet 104 proximally into the needle 102. This permits the user to manually extend and retract the stylet 104 with a desired level or force. In addition, the user may manually extract and retract the stylet 104 multiple times if more than one tissue sample is desired.

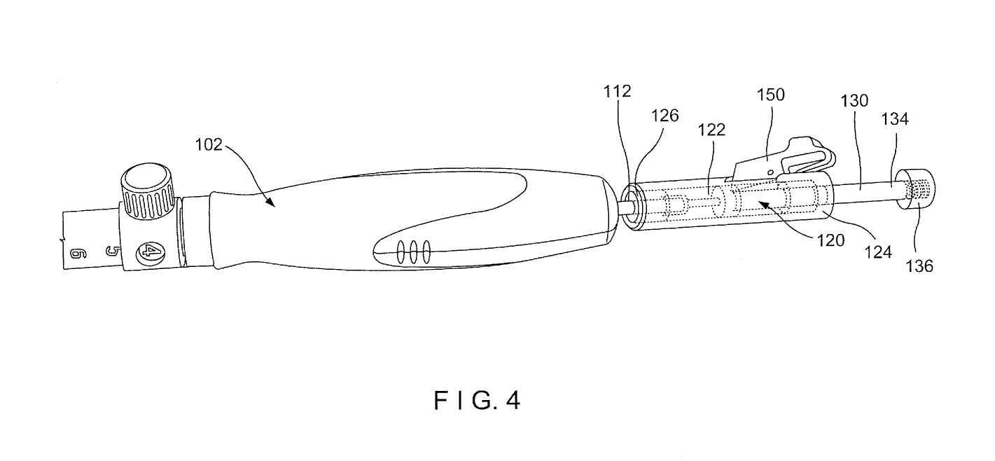

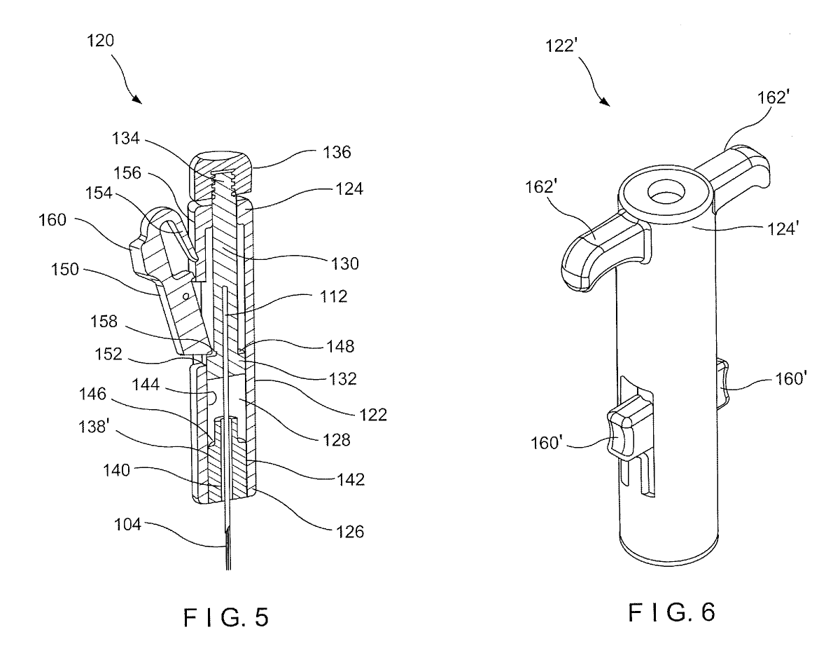

In an alternative embodiment, a mechanism for moving the stylet 104 between the tissue capture configuration and the closed configuration may be included as an add-on feature or as a separate device coupled to a handle of the device 100. For example, as shown in FIGS. 4-5, an actuating mechanism 120 may be attached to the proximal end 112 of a stylet 104 inserted through the lumen 108 of a needle 102 as shown in FIG. 3. The actuating mechanism 120, as shown in FIG. 4, comprises a housing 122, a piston 130 and a lever 150. The housing 122 extends from a proximal end 124 to a distal end 126 with a channel 128 extending therethrough. The piston 130 extends from a proximal end 134 to a distal end 132 with the distal end 132 movably housed within the channel 128 while a proximal end 134 extends proximally past the proximal end 124 of the housing 122 to engage a cap 136. The distal end 132 includes a shoulder 148 extending radially outward such that the distal end 132 of the piston is larger than a remaining portion of the piston 130 to engage an inner surface of the channel 128. The distal end 112 of the stylet 104 is fixed to the piston 130 such that moving the piston 130 longitudinally relative to the housing 122 moves the stylet 104 between the open and the closed configurations.

The actuating mechanism 120 further includes a female luer connection 138 configured for coupling to a male luer 138′ of the needle device. The stylet 104 passes through a lumen 140 of the male luer 138′ into the channel 128. For example, the female luer connection 138 may include a threaded inner surface 144 configured to engage a corresponding threaded outer surface 142 of the male luer 138′. It will be understood by those of skill in the art, however, that the luer 138 may engage the housing 122 in any number of ways. A mechanism (not shown) may be housed within the channel 128, between a proximal end 146 of the male luer 138′ and the distal end 132 of the piston 130, biasing the piston 130, and thus the stylet 104, toward the closed configuration. As would be understood by those skilled in the art, this mechanism may include a spring (e.g., a helical or a coil spring) biasing the piston 130. In an alternative embodiment, the mechanism may include a source of pressurized gas captured in, for example, a gas piston. It will be understood by those of skill in the art that any known mechanism may be employed to bias the piston 130, and thereby the stylet 104, toward the closed configuration.

The lever 150 is pivotably attached to a side of the housing 122 and extends through an opening 152 in a side of the housing 122. The lever 150 includes a biasing element 154 contacting an outer surface 156 of the housing 122 to bias the lever 150 toward a position angled relative to a longitudinal axis of the housing 122 so that an engaging element 158 of the lever extends through the opening 152 and engages a side of the piston 130. When the actuating mechanism 120 is in the closed configuration, the piston 130 is moved proximally through the channel 128 with the engaging element 158 of the lever 150 engaging the radially protruding distal end 132 of the piston. In this position, the lever 150 is rotated to a position substantially parallel to the longitudinal axis of the housing 122 (i.e., substantially parallel to the needle 102 extending therethrough). To move the device to the tissue capture configuration, a user pushes the cap 136 distally toward the proximal end 124 of the housing 122 moving piston 130 distally through the housing 122 until the engaging element 158 of the lever 150 moves out of contact with the radially protruding end 132 of the piston 130. At this point, the biasing element 154 rotates the lever 150 (counterclockwise as seen in FIG. 4) so that the engaging element 158 moves radially inward to engage the reduced diameter portion of the piston 130 seated proximally of the shoulder 148. The spring (not shown) in the channel 128 urges the piston 130 against the engaging element 158, locking the device in the tissue capture configuration. To withdraw the stylet 104 proximally into the closed configuration, the user simply rotates the lever 150 (clockwise as seen in FIG. 4) to disengage the engaging element 158 from the shoulder 148 so that the spring pushes the piston 130 proximally and the stylet 104 is drawn proximally into the needle 102 (i.e., into the closed configuration).

As described above, in the open configuration, the distal end 110 of the stylet 104 extends distally past the distal tip 106 of the needle 102 so that the arms 114 are spaced apart and in a configuration to receive tissue therebetween. Once target tissue 10 has been received in the channel 115 between the arms 114, a user presses a button 158 on the lever 150 to release the engaging element 156 from the shoulder 148 allowing the piston 130 to move proximally into the closed configuration. This draws the stylet 104 along with the collected target tissue 10 proximally into the lumen 108 of the needle 102.

As shown in FIG. 6, a housing 122′ of a device according to a further embodiment is substantially similar to the housing 122 of the actuating mechanism 120, but further includes ergonomic features to facilitate manipulation by a user. For example, the housing 122′ includes finger grips 160′ at a proximal end 124′ making the housing 122′ more easily gripped as the cap 136 is pressed into the tissue capture configuration. As opposed to the single button of the mechanism 120, the housing 122′ may include dual buttons 160′ positioned on opposing sides of the housing 122′ facilitating the such that the dual buttons 160′ may be easily pressed as the housing 122′ is gripped by the user. It will be understood by those of skill in the art that the dual buttons 160′ will work in substantially the same manner as described above for the button 160 of the lever 150.

Although the device 100 is described as being used with the actuating mechanism 120, it will be understood by those of skill in the art that the device 100 may be moved between the closed and tissue capture configurations using any actuating mechanism capable of drawing the stylet 104 into the lumen 108 of the needle 102.

It will be understood by those skilled in the art that various modifications and variations can be made in the structure and the methodology of the present invention, without departing from the spirit or scope of the invention. Thus, it is intended that the present invention cover the modifications and variations of this invention provided that they come within the scope of the appended claims and their equivalents.

What is claimed is:

1. A biopsy device, comprising:
a longitudinal element sufficiently flexible to permit insertion into a body via a natural body lumen, the longitudinal element extending from a proximal end to a distal end and including a lumen extending therethrough; and
a stylet slidably received within the lumen having a distal end split into a plurality of arms biased away from one another so that, when distal end of the stylet is advanced out of a distal end of the longitudinal element, the arms separate from one another into a tissue receiving configuration, the stylet including a tissue receiving space between the arms, wherein the stylet is coupled to a handle via which the stylet is drawn proximally into the lumen of the longitudinal element so that contact between the longitudinal element and the arms draws the arms toward one another into a tissue capture configuration in which tissue gripped therebetween.

2. The biopsy device of claim 1, wherein the handle includes an actuator coupled to the stylet for withdrawing the stylet proximally relative to the longitudinal element from the tissue receiving configuration to the tissue capture configuration.

3. The biopsy device of claim 1, wherein at least one of the arms include at least one tooth along an inner surface thereof for gripping the tissue captured between the arms.

4. The biopsy device of claim 1, wherein the stylet includes a pair of arms.

5. The biopsy device of claim 1, wherein at least one of the arms is treated with a coating for enhancing a gripping therebetween.

6. The biopsy device of claim 1, wherein at least one of the arms includes a recess along an inner surface thereof for receiving a tissue sample therein.

7. The biopsy device of claim 1, wherein the longitudinal element includes a distal facing tissue cutting edge past which the tissue receiving element moves when moved from the tissue receiving configuration to the tissue capture configuration to sever tissue received in the tissue receiving space from surrounding tissue.

8. The biopsy device of claim 1, wherein one of the longitudinal element and the stylet includes a tapered tissue piercing distal tip.

9. The biopsy device of claim 2, wherein the actuator includes a piston coupled to the one of the longitudinal element and the stylet for movement therewith, the piston being movably received within a handle of the device and being coupled to a biasing member biasing the piston toward a position corresponding to the tissue capture configuration.

10. The biopsy device of claim 9, wherein the one of the longitudinal element and the stylet is the stylet and wherein the actuator includes a latch mechanism for locking the stylet in the tissue capture configuration until released by a user to move the stylet proximally to the tissue capture configuration by the biasing member.

11. A method for obtaining a tissue sample, comprising:
inserting a biopsy device to a target location within a living body via a natural body lumen to a desired location adjacent to target tissue to be sampled, the biopsy device including a flexible longitudinal element having a lumen extending therethrough and a stylet slidably received within the lumen, the stylet including a distal end split into a pair of arms biased away from one another to form a tissue receiving space therebetween;
advancing the stylet distally relative to the longitudinal element so that, as the arms extend distally outside the longitudinal element, the arms are spread apart from one another to receive a portion of the target tissue within the tissue receiving space; and
retracting the stylet proximally into the longitudinal element, contact between the longitudinal element and the arms, drawing the arms into a tissue capture configuration in which the portion of target tissue is gripped therebetween, the portion of target tissue being severed from surrounding tissue and retained within the longitudinal element.

12. The method of claim 11, wherein the stylet is retracted into the longitudinal element by operating an actuator.

13. The method of claim 12, wherein the actuator is on a handle of the device which, when the longitudinal element is inserted to the desired location within the body, remains external to the body.

14. The method of claim 11, wherein at least one of the arms includes at least one tooth along an inner surface thereof for gripping the tissue captured between the arms.

15. The method of claim 11, wherein the stylet includes a pair of arms.

16. The method of claim 11, wherein at least one of the arms is treated with a coating for enhancing a gripping therebetween.

17. The method of claim 11, wherein at least one of the arms includes a recess along an inner surface thereof for receiving a tissue sample therein.

18. The method of claim 11, wherein a distal-facing edge of the longitudinal element forms a tissue cutting blade for severing the target tissue from the surrounding tissue.

19. The method of claim 12, wherein operation of the actuator releases a biasing member coupled to a piston coupled to the stylet so that movement of the piston draws the stylet proximally to the tissue capture configuration.

20. The method of claim 19, further comprising releasing a latch mechanism of the actuator which locks the stylet in the tissue capture configuration to move the stylet to the tissue capture configuration.

21. A biopsy device, comprising:
a longitudinal element sufficiently flexible to permit insertion into a body via a natural body lumen, the longitudinal element extending from a proximal end to a distal end and including a lumen extending therethrough;
a stylet slidably received within the lumen having a distal end split into a pair of arms biased away from one another so that, when distal end of the stylet is advanced out of a distal end of the longitudinal element, the arms separate from one another into a tissue receiving configuration, the stylet including a tissue receiving space between the arms, wherein the stylet is coupled to a an actuator so that upon activation, the actuator draws the stylet proximally into the lumen of the longitudinal element so that contact between the longitudinal element and the arms draws the arms toward one another into a tissue capture configuration in which tissue gripped therebetween; and
an actuator coupled to the stylet for withdrawing the stylet proximally relative to the longitudinal element from the tissue receiving configuration to the tissue capture configuration.
